SEQUENCE LISTING (81 sequences)

SEQ ID NO: 1 - RNA, 22 nt, Artificial Sequence
  miRNA-132 antisense strand
uaacagucuacagccauggucg

SEQ ID NO: 2 - RNA, 21 nt, Artificial Sequence
  miRNA-212 antisense strand
uaacagucuccagucacggcc

SEQ ID NO: 3 - RNA, 25 nt, Artificial Sequence
  miRNA-132 antisense strand
uaacagucuacagccauggucgccc

SEQ ID NO: 4 - RNA, 21 nt, Artificial Sequence
  miRNA-132  antisense strand
uaacagucuacagccaugguc

SEQ ID NO: 5 - RNA, 21 nt, Artificial Sequence
  miRNA-132  antisense strand
aacagucuacagccauggucg

SEQ ID NO: 6 - RNA, 23 nt, Artificial Sequence
  miRNA-132  antisense strand
uaacagucuacagccauggucgc

SEQ ID NO: 7 - RNA, 22 nt, Artificial Sequence
  miRNA-132  antisense strand
aacagucuacagccauggucgc

SEQ ID NO: 8 - RNA, 21 nt, Artificial Sequence
  miRNA-132  antisense strand
acagucuacagccauggucgc

SEQ ID NO: 9 - RNA, 22 nt, Artificial Sequence
  miRNA-132  antisense strand
uaacagucuacagccauggucg

SEQ ID NO: 10 - RNA, 20 nt, Artificial Sequence
  miRNA-132  antisense strand
uaacagucuacagccauggu

SEQ ID NO: 11 - RNA, 19 nt, Artificial Sequence
  miRNA-132  antisense strand
uaacagucuacagccaugg

SEQ ID NO: 12 - RNA, 18 nt, Artificial Sequence
  miRNA-132  antisense strand
uaacagucuacagccaug

SEQ ID NO: 13 - RNA, 16 nt, Artificial Sequence
  miRNA-132  antisense strand
uaacagucuacagcca

SEQ ID NO: 14 - RNA, 22 nt, Artificial Sequence
  miRNA-132  antisense strand
uaacagucuacagccauggucg

SEQ ID NO: 15 - RNA, 20 nt, Artificial Sequence
  miRNA-132  antisense strand
acagucuacagccauggucg

SEQ ID NO: 16 - RNA, 22 nt, Artificial Sequence
  miRNA-132  antisense strand
uaacggucuacagccauggucg

SEQ ID NO: 17 - RNA, 21 nt, Artificial Sequence
  miRNA-132  antisense strand
uaacagucuacagccaugguc

SEQ ID NO: 18 - RNA, 22 nt, Artificial Sequence
  miRNA-132  antisense strand
uaacagucuacagccauggucg

SEQ ID NO: 19 - RNA, 22 nt, Artificial Sequence
  miRNA-132  antisense strand
uaacagucuacagccguggucg

SEQ ID NO: 20 - RNA, 21 nt, Artificial Sequence
  miRNA-132  antisense strand
uaacagucuacagccaugguc

SEQ ID NO: 21 - RNA, 21 nt, Artificial Sequence
  miRNA-132  antisense strand
aacagucuacagccauggucg

SEQ ID NO: 22 - RNA, 22 nt, Artificial Sequence
  miRNA-132  antisense strand
uaacagucuacagccauggccg

SEQ ID NO: 23 - RNA, 23 nt, Artificial Sequence
  miRNA-132  antisense strand
aacagucuacagccauggucgcc

SEQ ID NO: 24 - RNA, 22 nt, Artificial Sequence
  miRNA-132  antisense strand
uaacagucuacagccauggucg

SEQ ID NO: 25 - RNA, 23 nt, Artificial Sequence
  miRNA-132  antisense strand
uaacagucuacagccauggucgu

SEQ ID NO: 26 - RNA, 17 nt, Artificial Sequence
  miRNA-132  antisense strand
gucuacagccauggucg

SEQ ID NO: 27 - RNA, 22 nt, Artificial Sequence
  miRNA-132  antisense strand
  misc_feature 14: n = A,T,C or G
uaacagucuacagncauggucg

SEQ ID NO: 28 - RNA, 22 nt, Artificial Sequence
  miRNA-132  antisense strand
uaacagucuacagccauggucg

SEQ ID NO: 29 - RNA, 21 nt, Artificial Sequence
  miRNA-132  antisense strand
uaacagucuacagccaugguc

SEQ ID NO: 30 - RNA, 21 nt, Artificial Sequence
  miRNA-132  antisense strand
acagucuacagccauggucgc

SEQ ID NO: 31 - RNA, 22 nt, Artificial Sequence
  miRNA-132  antisense strand
aacagucuccagccauggucgc

SEQ ID NO: 32 - RNA, 22 nt, Artificial Sequence
  miRNA-132  antisense strand
uaacagucuacagccauggucg

SEQ ID NO: 33 - RNA, 22 nt, Artificial Sequence
  miRNA-132  antisense strand
caacagucuacagccauggucg

SEQ ID NO: 34 - RNA, 21 nt, Artificial Sequence
  miRNA-132  antisense strand
acaggcuacagccauggucgc

SEQ ID NO: 35 - RNA, 22 nt, Artificial Sequence
  miRNA-132  antisense strand
uaacagucuacagccauggucg

SEQ ID NO: 36 - RNA, 22 nt, Artificial Sequence
  miRNA-132  antisense strand
  misc_feature 1: n = A,T,C or G
naacagucuacagccauggucg

SEQ ID NO: 37 - RNA, 25 nt, Artificial Sequence
  miRNA-132  sense strand
gggcgaccauggcuguagacuguua

SEQ ID NO: 38 - RNA, 22 nt, Artificial Sequence
  miRNA-132  sense strand
accguggcuuucgauuguuacu

SEQ ID NO: 39 - RNA, 21 nt, Artificial Sequence
  miRNA-132  sense strand
accguggcuuucgauuguuac

SEQ ID NO: 40 - RNA, 20 nt, Artificial Sequence
  miRNA-132  sense strand
accguggcuuucgauuguua

SEQ ID NO: 41 - RNA, 21 nt, Artificial Sequence
  miRNA-132  sense strand
aaccguggcuuucgauuguua

SEQ ID NO: 42 - RNA, 19 nt, Artificial Sequence
  miRNA-132  sense strand
accguggcuuucgauuguu

SEQ ID NO: 43 - RNA, 18 nt, Artificial Sequence
  miRNA-132  sense strand
accguggcuuucgauugu

SEQ ID NO: 44 - RNA, 19 nt, Artificial Sequence
  miRNA-132  sense strand
aaccguggcuuucgauugu

SEQ ID NO: 45 - RNA, 22 nt, Artificial Sequence
  miRNA-132  sense strand
aaccguggcuuucgauuguuac

SEQ ID NO: 46 - RNA, 20 nt, Artificial Sequence
  miRNA-132  sense strand
aaccguggcuuucgauuguu

SEQ ID NO: 47 - RNA, 21 nt, Artificial Sequence
  miRNA-132  sense strand
cccguggcuuucgauuguuac

SEQ ID NO: 48 - RNA, 17 nt, Artificial Sequence
  miRNA-132  sense strand
accguggcuuucgauug

SEQ ID NO: 49 - RNA, 23 nt, Artificial Sequence
  miRNA-132  sense strand
aaccguggcuuucgauuguuacu

SEQ ID NO: 50 - RNA, 22 nt, Artificial Sequence
  miRNA-132  sense strand
accguggcuuucgauuguuacc

SEQ ID NO: 51 - RNA, 21 nt, Artificial Sequence
  miRNA-132  sense strand
accguggcuuucgauuguuac

SEQ ID NO: 52 - RNA, 22 nt, Artificial Sequence
  miRNA-132  sense strand
accguggcuuucgauuguuacu

SEQ ID NO: 53 - RNA, 21 nt, Artificial Sequence
  miRNA-132  sense strand
accgugacuuucgauuguuac

SEQ ID NO: 54 - RNA, 18 nt, Artificial Sequence
  miRNA-132  sense strand
aaccguggcuuucgauug

SEQ ID NO: 55 - RNA, 17 nt, Artificial Sequence
  miRNA-132  sense strand
accguggcuuucgauug

SEQ ID NO: 56 - RNA, 19 nt, Artificial Sequence
  miRNA-132  sense strand
aaccguggcuuucgauugu

SEQ ID NO: 57 - RNA, 20 nt, Artificial Sequence
  miRNA-132  sense strand
accguggcuuucgauuguua

SEQ ID NO: 58 - RNA, 21 nt, Artificial Sequence
  miRNA-132  sense strand
accgcggcuuucgauuguuac

SEQ ID NO: 59 - RNA, 22 nt, Artificial Sequence
  miRNA-132  sense strand
  misc_feature 1: n = A,T,C or G
nccguggcuuucgauuguuacu

SEQ ID NO: 60 - RNA, 20 nt, Artificial Sequence
  miRNA-132  sense strand
caccguggcuuucgauuguu

SEQ ID NO: 61 - RNA, 23 nt, Artificial Sequence
  miRNA-212  sense strand
accuuggcucuagacugcuuacu

SEQ ID NO: 62 - RNA, 22 nt, Artificial Sequence
  miRNA-212  sense strand
accuuggcucuagacugcuuac

SEQ ID NO: 63 - RNA, 24 nt, Artificial Sequence
  miRNA-212  sense strand
accuuggcucuagacugcuuacug

SEQ ID NO: 64 - RNA, 23 nt, Artificial Sequence
  miRNA-212  sense strand
ccuuggcucuagacugcuuacug

SEQ ID NO: 65 - RNA, 23 nt, Artificial Sequence
  miRNA-212  sense strand
cccuuggcucuagacugcuuacu

SEQ ID NO: 66 - RNA, 21 nt, Artificial Sequence
  miRNA-212  sense strand
ccuuggcucuagacugcuuac

SEQ ID NO: 67 - RNA, 21 nt, Artificial Sequence
  miRNA-212  sense strand
accuuggcucuagacugcuua

SEQ ID NO: 68 - RNA, 20 nt, Artificial Sequence
  miRNA-212  sense strand
caccccgcccggacacggac

SEQ ID NO: 69 - RNA, 21 nt, Artificial Sequence
  miRNA-212  antisense strand
uaacagucuccagucacggcc

SEQ ID NO: 70 - RNA, 22 nt, Artificial Sequence
  miRNA-212  antisense strand
uaacagucuccagucacggcca

SEQ ID NO: 71 - RNA, 23 nt, Artificial Sequence
  miRNA-212  antisense strand
uaacagucuccagucacggccac

SEQ ID NO: 72 - RNA, 22 nt, Artificial Sequence
  miRNA-212  antisense strand
guaacagucuccagucacggcc

SEQ ID NO: 73 - RNA, 21 nt, Artificial Sequence
  miRNA-212  antisense strand
uaacagucuccagucacggcc

SEQ ID NO: 74 - RNA, 22 nt, Artificial Sequence
  miRNA-212  antisense strand
uaacagucuccagucacggcca

SEQ ID NO: 75 - RNA, 21 nt, Artificial Sequence
  miRNA-212  antisense strand
uaacagucuccagucacggcc

SEQ ID NO: 76 - RNA, 20 nt, Artificial Sequence
  miRNA-212  antisense strand
uaacagucuccagucacggc

SEQ ID NO: 77 - RNA, 20 nt, Artificial Sequence
  miRNA-212  antisense strand
uaacagucuccagucacggc

SEQ ID NO: 78 - RNA, 21 nt, Artificial Sequence
  miRNA-212  antisense strand
guaacagucuccagucacggc

SEQ ID NO: 79 - RNA, 22 nt, Artificial Sequence
  miRNA-212  antisense strand
uaacagucuccagucacggcca

SEQ ID NO: 80 - RNA, 24 nt, Artificial Sequence
  miRNA-212  antisense strand
uaacagucuccagucacggccaga

SEQ ID NO: 81 - RNA, 21 nt, Artificial Sequence
  miRNA-212  antisense strand
uaacagucuccagucacgucc

(19) United States
(12) Patent Application Publication
(10) Pub. No.: US 2017/0246200 A1
(43) Pub. Date: Aug. 31, 2017

(54) MICRORNA-132/212 FOR THE TREATMENT OF NEURODEGENERATIVE DISORDERS

(71) Applicant: UNIVERSITÉ LAVAL, Québec (CA)

(72) Inventor: Sébastien HEBERT, Québec (CA)

(73) Assignee: UNIVERSITÉ LAVAL, Québec, QC (CA)

(21) Appl. No.: 15/513,181

(22) PCT Filed: Sep. 22, 2015
(86) PCT No.: PCT/CA2015/050931

§ 371 Date: Mar. 22, 2017

Related U.S. Application Data
(60) Provisional application No. 62/053,308, filed on Sep. 22, 2014.

(51) Int. Cl. A61K 31/7105; A61K 48/00; C12Q 1/68; C12N 15/113

(52) U.S. Cl. CPC A61K 31/7105; C12N 15/1138; A61K 48/00; C12Q 1/68; C12Q 2600/158; G01N 2800/2814

(57) ABSTRACT

The present relates to the use of miRNA-132/212 mimics or activators comprising a doubled stranded ribonucleic acid molecule comprising a seed region sequence of miRNA-132 or miRNA-212, a spacer, a stabilizing sequence, and a carrier for the treatment of neurodegenerative disorders, including Alzheimer's disease, Tauopathies, Amyotrophic lateral sclerosis, Parkinson's disease, frontotemporal dementia, prion's disease, mild cognitive impairment and Huntington's disease.

### CROSS-REFERENCE TO RELATED APPLICATIONS

This application includes a sequence listing in electronic format which is being concurrently filed herewith. This application also claims priority from U.S. provisional application Ser. No. 62/053,308 filed on Sep. 22, 2014. The content of the sequence listing and of the priority application is herewith incorporated in its entirety.

### TECHNICAL FIELD

The present invention relates to mimics or activators of miRNA-132 and/or miRNA-212 molecules useful for treating neurodegenerative disorders.

### BACKGROUND ART

Neurodegenerative disorders are diseases or conditions associated with the progressive loss of structure or function of the brain, including death of neurons. Many neurodegenerative diseases including Alzheimer's disease, tauopathies, Amyotrophic lateral sclerosis, Parkinson's disease, frontotemporal dementia, prion's disease, and Huntington's disease occur as a result of neurodegenerative processes and dementia. There are many parallels between different neurodegenerative disorders including atypical protein assemblies as well as induced cell death.

Alzheimer's disease is a progressive age-related dementia were amyloid plaques, neurofibrillary tangles, inflammation and vascular amyloidopathy is observed in the brain of inflected patients. The amyloid plaques and neurofibrillary tangles are characterized by the deposition of insoluble protein aggregates in the brain. Neurofibrillary tangles are composed of paired helical filaments, which comprise hyperphosphorylated forms of the microtubule-associated protein Tau. The main constituents of amyloid plaques are the ˜4 kD amyloid-beta (Aβ) peptides, which are a proteolytic product of the Amyloid-β precursor protein (APP). APP is cleaved by the β-site amyloid precursor protein-cleaving enzyme 1 (BACE1) and γ-secretase complex (PSEN, APH1, PEN2, NICASTRIN) to generate Aβ peptides.

Tauopathies are referred as a class of neurodegenerative diseases associated with the pathological aggregation of Tau protein in the human brain. These include, but are not limited to, Alzheimer's disease, Pick's disease, Supranuclear palsy, Corticobasal degeneration, Frontotemporal dementia and parkinsonism linked to chromosome 17, Tangle-predominant dementia, Lytico-Bodig disease, and Dementia pugilistica.

Accumulating evidence suggests that changes in gene expression and/or protein translation may contribute to neurodegenerative diseases such as Alzheimer's disease. For instance, duplication of the APP gene, which increases APP protein levels, can cause familial (genetic) Alzheimer's disease. Polymorphisms in the APP promoter, which increase APP transcription, have also been associated with Alzheimer's disease. High levels of BACE1/β-secretase protein levels in sporadic late-onset may be explained, at least in part, by abnormal translation of BACE1 messenger RNA (mRNA).

microRNAs (miRNAs) play an essential role in post-transcriptional gene expression in all living organisms. Following transcription, initial processing and export into the cytoplasm, miRNA precursors (˜70 nt in length) are cleaved by the ribonuclease Dicer to yield mature (˜21 nt in length) miRNAs. These short RNAs function as part of the RNA-induced silencing complex (RISC) to negatively regulate gene expression. This is done through binding of the RISC complex mainly to the 3′untranslated region (3′UTR) of target messenger RNAs (mRNAs), leading to their translational repression or degradation. The miRNA “seed” sequence (positions 2-8 in the miRNA) is important for mRNA targeting and functions. Accordingly, the main function of miRNAs is to repress protein levels.

Alterations in the miRNA network could contribute to risk for Alzheimer's disease. Studies in humans have detected changes in miRNA expression patterns in Alzheimer brain and peripheral system. Several affected miRNAs have been implicated in the regulation of key genes involved in Alzheimer's disease and related disorders, including APP, BACE1 and Tau.

Drugs that inhibit the degradation of acetylcholine within synapses are the current strategy used to treat symptoms of Alzheimer's disease. Donepezil, rivastigmine, and galantamine are example of such treatment that are safe but have potentially troublesome cholinergic side effects, including nausea, anorexia, diarrhea, vomiting, and weight loss. These drugs improve temporarily cognition in certain, but not all, patients. These drugs do not halt or reverse the neurodegeneration or pathologies causing dementia.

Most (>95%) Alzheimer's disease drugs developed so far in vitro have failed in clinical trials. Examples include anti-Aβ antibodies bapineuzumab and solanezumab used in immunotherapies.

There is still a need to be provided with therapeutic approaches to treat or prevent neurodegenerative disease such as Alzheimer's disease.

### SUMMARY

In accordance with the present description there is now provided a pharmaceutical composition for treating a neurodegenerative disorder in a patient comprising a doubled stranded ribonucleic acid molecule comprising a seed region sequence of miRNA-132 or miRNA-212, a spacer, a stabilizing sequence, and a carrier.

In accordance with the present description there is also provided a method for treating a neurodegenerative disorder in a patient comprising administering to the patient a doubled stranded ribonucleic acid molecule comprising a seed region sequence of miRNA-132 or miRNA-212 and a stabilizing sequence.

In an embodiment, the spacer sequence comprises a sequence 3′ of the miRNA-132 or -212 seed sequence.

In an embodiment, the stabilizing sequence consists of the RNAi-cap™ sequence.

In another embodiment, the doubled stranded ribonucleic acid molecule comprises one strand consisting of SEQ ID NO: 1 to 81.

In a further embodiment, the doubled stranded ribonucleic acid molecule comprises one strand consisting of SEQ ID NO: 3 and a second strand consisting of SEQ ID NO: 37.

In an embodiment, the doubled stranded ribonucleic acid molecule described herein further comprises uracil.

In an embodiment, the doubled stranded ribonucleic acid molecule described herein further comprises at least one cytosine modified at the 5′-position.

In an embodiment, the doubled stranded ribonucleic acid molecule described herein further comprises methylcytosine, 5-(2-amino)propyluracil, 5-bromouracil, 8-bromoguanine, 7-deaza-adenine or N6 alkyl-adenine.

In an embodiment, the doubled stranded ribonucleic acid molecule described herein further comprises at least one sugar-modified block.

In an embodiment, the doubled stranded ribonucleic acid molecule described herein further comprises at least one sugar-modified ribonucleotide building block.

In an embodiment, the doubled stranded ribonucleic acid molecule described herein further comprises at least one of LNA or a morpholino nucleotide.

In an embodiment, the doubled stranded ribonucleic acid molecule described herein further comprises at least one phosphodiester group.

In an embodiment, the doubled stranded ribonucleic acid molecule described herein further comprises at least one phosphorothioation modification.

In an embodiment, the doubled stranded ribonucleic acid molecule described herein further comprises at least one phosphoroamidate deoxyribonucleotide moiety.

In an embodiment, the doubled stranded ribonucleic acid molecule described herein further comprises at least one ribonucleotide moiety that is substituted at the 2′ position.

In an embodiment, the doubled stranded ribonucleic acid molecule described herein further comprises at least one methylene bridge.

In an embodiment, the doubled stranded ribonucleic acid molecule described herein further comprises at least one 2′-fluororibonucleotide moiety.

In another embodiment, the composition described herein further comprises cholesterol, penetratin, transportan, or TAT peptide.

In a supplemental embodiment, the carrier is a saline solution, glucose, or a lipid-based solution.

In another embodiment, the carrier is LipofectAMINE or in vivo-jetPEI.

In an embodiment, the patient is a mammal.

In an embodiment, the mammal is a mouse, a primate or a human.

In an embodiment, the neurodegenerative disorder is Alzheimer's disease, tauopathy, Amyotrophic lateral sclerosis, Parkinson's disease, frontotemporal dementia, prion's disease, mild cognitive impairment or Huntington's disease.

In an embodiment, the neurodegenerative disorder is Alzheimer's disease.

In a further embodiment, the composition described herein further comprises a natural or synthetic compound.

In an embodiment, the natural or synthetic compound increases endogenous miRNA-132 or miRNA-212 expression levels in the central nervous system.

In an embodiment, the natural or synthetic compound is Leptin or Luteolin.

In another embodiment, the composition described herein further comprises an anti-Alzheimer's compound.

In another embodiment, the anti-Alzheimer's compound is Donepezil, rivastigmine, galantamine, a cholinesterase inhibitor, memantine, vitamin E, an anti-Aβ antibody, an omega-3 fatty-acid, or stem cells.

In another embodiment, the composition described herein is formulated for an oral or systemic administration.

In an embodiment, the composition described herein is formulated for a systemic administration is an enteral or parenteral administration.

In an embodiment, parenteral administration is an intravenous administration, and intramuscular administration, or a subcutaneous injection.

In an embodiment, the composition described herein is formulated for an intrathecal administration.

In an embodiment, the composition described herein is formulated for an intracerebroventricular administration.

In an embodiment, the method described herein further comprises administering with the doubled stranded ribonucleic acid molecule a natural or synthetic compound.

In an embodiment, the doubled stranded ribonucleic acid molecule is administered via an oral or systemic administration.

In an embodiment, the doubled stranded ribonucleic acid molecule is administered via an intrathecal administration.

In an embodiment, the doubled stranded ribonucleic acid molecule is administered via an intracerebroventricular administration.

### BRIEF DESCRIPTION OF THE DRAWINGS

Reference will now be made to the accompanying drawings.

FIG. 1 illustrates in (a) a Western blot analysis of Tau protein expression and phosphorylation measured from cortex lysates of 6 month-old wildtype control and miRNA-132/212 knockout (KO) mice (n=8 animals per genotype, mixed gender); in (b, c) a histogram showing the quantification of Tau protein expression and phosphorylation measured in (a) wherein total Tau (T-Tau) and GAPDH were used as normalizing controls; in (d) a histogram showing the quantification of Tau mRNA measured in (a) wherein GAPDH was used as normalizing control; in (e) a bioinformatics analysis showing the predicted miRNA-132/212 binding within the 3′untranslated region of Tau mRNA; in (f) a histogram showing that miRNA-132 mimics directly target the 3′untranslated region of Tau in a luciferase reporter assay, compared to controls consisting of either a mutated miRNA-132 binding site in Tau mRNA, a scrambled miRNA mimic, or the unrelated miRNA-195; in (g) a Western blot analysis of Tau protein expression in N2A cells treated with miRNA-132 mimics, compared to a scrambled control; in (h) a histogram showing the quantification of Tau mRNA measured in (g) wherein GAPDH was used as normalizing control, standard error of the means (SEM) and molecular markers are shown, and statistical significances were determined by Student's unpaired t-test (*P<0.05, ***P<0.0001).

FIG. 2 illustrates a Western blot analysis of soluble (a) and sarkosyl-insoluble (d) Tau protein fractions from cortex lysates of 6 month-old 3×Tg-AD control and 3×Tg-AD miRNA-132/212 KO (n=8 animals/genotype), wherein total Tau (T-Tau), GAPDH, and Hsp40 were used as normalization controls; in (b, c, e, f) are histograms showing the quantification of Tau protein expression and phosphorylation measured in (a, d) wherein total Tau (T-Tau) and GAPDH were used as normalizing controls; in (g) a immunohistochemistry analysis of Tau neurofibrillary tangles in 18 month-old 3×Tg-AD control and 3×Tg-AD miRNA-132/212 KO (n=4 animals/genotype), standard error of the means (SEM) are shown, and statistical significances were determined by Student's unpaired t-test (*P<0.05), (**P<0.01), (***P<0.001).

FIG. 3 illustrates an immunoblot (a) and a histogram analysis (b) of APP and BACE1 from cortex lysates of 6 month-old wildtype control and miRNA-132/212 KO mice (n=8 animals/genotype, mixed gender), wherein APP full length (APP), APP C-terminal fragments (APP-CTFs) and BACE1 were detected, and GAPDH was used as normalization control; in (c) a Western blot analysis of HEK293-APPSwe cells transfected with miRNA-132 mimics or scrambled control, wherein APP full length, APP C-terminal fragments and BACE1 are shown, and normalized to GAPDH; in (d) a Western blot analysis of BACE1 of cortex lysates of 12 month-old 3×Tg-AD control mice treated with miRNA-132 mimics or vehicle (n=10 animals/genotype, mixed gender), wherein GAPDH was used as normalization control; in (e) Aβ40 and Aβ42 peptides from supernatants of transfected HEK293-APPswe cells were measured by human-specific Aβ ELISA kits (n=2 in triplicate), wherein absolute quantifications (pg/mL) are shown; in (f) a immunoblot analysis of overexpressed human BACE1 protein with transfected miRNA-132 mimics in N2A cells (n=3 in triplicate), wherein GAPDH used as normalizing control, standard error of the means (SEM), and statistical significances were determined by Student's unpaired t-test (*P<0.05, ***P<0.0001).

FIG. 4 illustrates a Western blot analysis and histogram of APP full-length and BACE1 of cortex lysates from 3×Tg-AD control and 3×Tg-AD miRNA-132/212 KO mice (n=8 animals/genotype, mixed gender) from 6 month-old (a, b) and from 12 month-old (c, d) animals, wherein GAPDH was used as normalizing control, standard error of the means (SEM) are shown; in (e) representing the measured Aβ40 peptides and Aβ42 peptides from cortex of 12 month-old 3×Tg-AD control and 3×Tg-AD miRNA-132/212 KO mice in soluble and sarkosyl insoluble fractions using ELISA kits. For ELISA data, absolute quantifications (pg/mL) are represented, and statistical significance was determined by Student's unpaired t-test (*P<0.05), in (f) illustrates an immunohistochemical analysis of increased Aβ plaques in 18-month-old 3×Tg-AD miRNA-132/212 KO mice compared to 3×Tg-AD control mice.

FIG. 5 illustrates the Barnes maze learning paradigms of naive 6-month-old (a, b, c, d) and 12-month-old (e, f, g, h) 3×Tg-AD control mice and 3×Tg-AD/miRNA-132/212 KO mice (N=12/group, mixed gender); in (l, j, k, l) illustrates the Barnes maze learning paradigms of experienced 12-month-old 3×Tg-AD control mice and 3×Tg-AD/miRNA-132/212 KO mice (N=12/group, mixed gender), wherein the naive mice had no prior experience in the Barnes maze or any other behavioral tasks, and the experienced mice performed the Barnes maze test (learning phase) 6 months earlier, and statistical significances were assessed by two-way ANOVA (repeated measures) with Bonferroni multiple comparison test, where *=p<0.05, **=p<0.01, and ***=p<0.001.

FIG. 6 illustrates in (a) the experimental paradigms of the Barnes maze used; in (b, d) illustrates the Learning phase of 12 month-old 3×Tg-AD control mice treated with miRNA-132 mimics compared to 3×Tg-AD control mice treated with saline (N=11/group, mixed gender); in (c, e) illustrates the Probe trials in the Barnes maze performed at day 1, week 1, week 2 and week 3 after the Learning phase; in (f, g, h) illustrates a Western blot and histogram analysis of Tau expression and phosphorylation in cortical brain samples from treated mice in (a), wherein GAPDH and total Tau (T-tau) were used as normalization controls, and statistical significances was done by Student's unpaired t-test, where *=p<0.05, **=p<0.01, and ns=p>0.05.

FIG. 7 illustrates in (a, b, c) an immunofluorescence analysis of fluorescent miRNA-132 mimics injected in 12 month old wildtype mice; in (d) is shown the relative expression of miRNA-132 measured by qRT-PCR in the cortex, the hippocampus, the cerebellum, the striatum, the brainstem, and the cerebellum of 3×Tg-AD control mice injected with miRNA-132 mimics (N=3/group), wherein control animals received saline.

FIG. 8 illustrates in (a) the relative miRNA-132 expression levels in various brain regions of post-mortem tissues in non-demented control (N=13) and Alzheimer's disease individuals (N=10) from the Douglas Bell Canada Brain Bank cohort, wherein let-7a was used as normalization control; in (b) is shown the relative miRNA-132 expression levels in non-demented control (N=11), mild cognitive impairment (MCI) (N=10), and Alzheimer's disease (AD) (N=11) groups from the Religious Orders Study cohort, where statistical significances were obtained by a Mann-Whitney test; in (c) is shown a correlation analysis of miRNA-132 expression levels and insoluble Tau in the Religious Orders Study cohort; is shown a correlation of miRNA-132 expression levels with (d) Mini-Mental State Examination (MMSE), (e) working memory, (f) perceptual speed, (g) episodic memory, (h) semantic memory, (i) visuospatial ability, and (j) global cognitive scores in all individuals the Religious Orders Study cohort, and statistical significances were determined using a linear regression analysis.

FIG. 9 illustrates in (a) an immunoblot analysis of APP, BACE1 and Tau protein measured from hippocampal extracts of 3-6 month-old wildtype mice treated with chemically-modified miRNA-16 mimics (miR-16 mod.) harboring 2′O-Me modifications on both sense and antisense stands, whereas control mice received saline; in (b) represents a qRT-PCR analysis of miR-16-mod.-treated mice, indicating that miRNA-16 expression levels are not significantly increased following treatment (n=8/group, mixed gender), and data are shown as mean±SEM.

FIG. 10 illustrates a Western blot analysis of (a) APP, BACE1 and Tau protein in N2A cells treated miRNA-132 mimics diluted in saline 0.9% (carrier example #1) or LipofectAMINE® (carrier example #2); in (b) represents a qRT-PCR analysis of miRNA-132 expression levels measured in the brains of 2-6 month-old wildtype mice treated with miRNA-132 mimics diluted in 5% glucose (carrier example #3) or in vivo-jetPEI® (carrier example #4) (average fold enrichment over endogenous miRNA-132 of N=5/group).

FIG. 11 illustrates in (a, b) a graphical representation of experiments performed in macaques (N=4), wherein animals received miRNA-132 or scrambled mimics, and CSF samples were taken before and after treatment; in (c) are shown ELISA measurements of CSF Aβ40 peptides and Aβ42 peptides (ratio) and total Tau.

### DETAILED DESCRIPTION

It is provided a composition comprising a mimic miRNA-132 and/or miRNA-212 sequence. The composition described herein is particularly useful for treating neurodegenerative disorders.

miRNA-132 and miRNA-212 are expressed as part of the miRNA-132/212 cluster that is located on chromosome 17 in humans (chromosome 11 in mice) (miRBase.org). Both mature sequence of miRNA-132 and miRNA-212 share the same miRNA seed sequence, and thus presumably target similar genes:

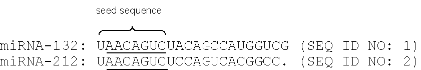

miRNA-132 expression is enriched in the brain, especially in neurons, while miRNA-212 expression is only weakly expressed in all tissues. It has been shown that miRNA-132 is the major functional species in vivo in the brain. Several brain-related functions have been attributed to miRNA-132. Overexpression of miRNA-132 in neuronal cultures caused a marked increase in neurite outgrowth. miRNA-132 is also directly implicated in activity-dependent spine formation. Transgenic mice overexpressing miRNA-132 have a marked increase in dendritic spine density, impairments in a novel object recognition memory test, as well as changes in hippocampal-dependent learning and memory. miRNA-132 overexpression modulates short-term synaptic plasticity in hippocampal cultures. miRNA-132/212 adult knockout mice have impaired learning and memory.

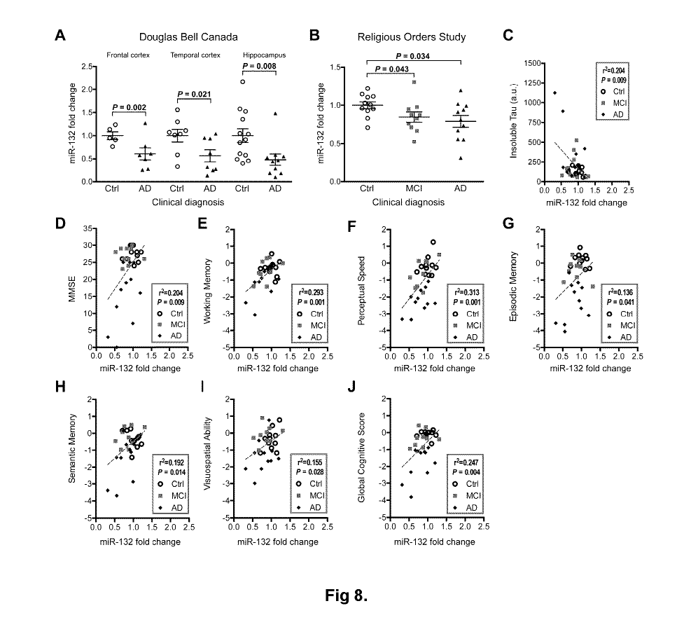

miRNA-132/212 is downregulated in Alzheimer's disease patients, and its expression levels correlate with Tau deposition (FIG. 8). miRNA-132 and miRNA-212 are downregulated in frontotemporal dementia and related Tauopathies. miRNA-132 and miRNA-212 are downregulated in the brain of α-synuclein (A30P)-transgenic mice, a model of Parkinson's disease. miRNA-132 and miRNA-212 are both deregulated in schizophrenia and bipolar disorders.

Based on the prediction that loss of miRNA-132 and -212 in the brain induces and/or exacerbate neurodegenerative diseases such as Alzheimer's disease, it is provided a replacement therapy based on the administration of a composition comprising a mimic or activator of miRNA-132 and/or miRNA-212 that compensate for the loss of miRNA-132 and/or -212 in affected patients. Contrary to the therapy proposed in International application publication no. WO 21013/034653 wherein inhibitors of miRNA-132 and/or of miRNA-212 are described, the present description provides a mean to supplement and compensate for the loss of miRNA-132 and/or 212.

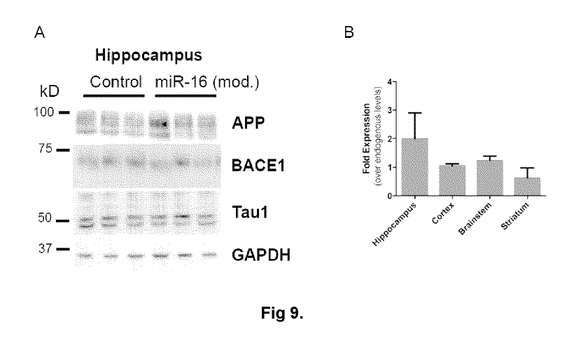

In addition, contrary to previous disclosure, such as for example WO 2015/006705, wherein all known miRNAs were thought to effectively regulate the expression of Tau, taken together, the results disclosed herein provide substantial evidence that miRNA-132 and miRNA-212 play an important role in neuronal homeostasis, learning, and memory formation, all of which are affected in neurodegenerative disorders such as Alzheimer's disease. As seen in FIG. 9 wherein the use of a miRNA-16 mimic did not increase the miRNA-16 expression levels or affect key Alzheimer's disease genes APP, BACE1, and Tau following treatment. The mimic miRNA-132 or miRNA-212 encompassed herein were effectively enabled for the use of such mimics for restoring/compensate for the loss of miRNA-132 and/or 212.

The mimic miRNA-132 or miRNA-212 encompassed herein is a doubled stranded ribonucleic acid molecule comprising the seed sequence of miRNA-132 or miRNA-212, a spacer and a stabilizing sequence.

As encompassed herein, the stabilizing sequence improves stability in body fluids. For example, the stabilizing sequence encompassed herein, but not limited to, is the RNAi-cap™ sequence (Riboxx GmbH). This stabilizing sequence induces directional modulation of RNA-Induced Silencing Complex (RISC) unwinding of miRNA mimic duplex, enhancing loading of the mature miRNA to the RISC complex. The latter sequence also protects miRNA mimics from serum degradation and reduces off-target effects.

Accordingly, the mimic miRNA encompassed herein comprises the following schematic representation:

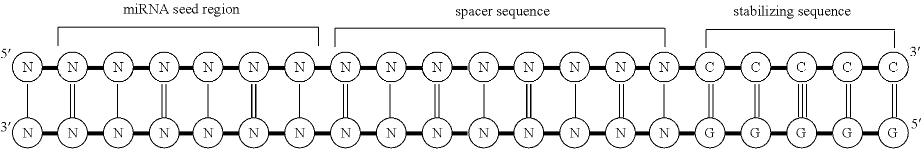

Minimally, the mimic miRNA encompassed herein comprises the miRNA seed region of miRNA-132 or 212 and a stabilizing sequence. Seed-targeting 8-mer injection in primates were sufficient to derepress miRNA targets. Accordingly, the use of a mimic miRNA comprising the miRNA seed region of miRNA-132 or -212 and a stabilizing sequence should be sufficient to compensate for the loss of miRNA-132 and/or -212 in patients, without loss of specificity or display toxicity.

Preferably, the mimic miRNA encompassed herein comprises at least one strand having the following sequences:

| Sequence | miRNA | SEQ ID No. |
|---|---|---|
| 5′-UAACAGUCUACAGCCAUGGUCG-3′ | miRNA-132 antisense strand | SEQ ID NO: 1 |
| 5′-UAACAGUCUACAGCCAUGGUCGCCC-3′ | miRNA-132 antisense strand | SEQ ID NO: 3 |
| 5′-UAACAGUCUACAGCCAUGGUC-3′ | miRNA-132 antisense strand | SEQ ID NO: 4 |
| 5′-AACAGUCUACAGCCAUGGUCG-3′ | miRNA-132 antisense strand | SEQ ID NO: 5 |
| 5′-UAACAGUCUACAGCCAUGGUCGC-3′ | miRNA-132 antisense strand | SEQ ID NO: 6 |
| 5′-AACAGUCUACAGCCAUGGUCGC-3′ | miRNA-132 antisense strand | SEQ ID NO: 7 |
| 5′-ACAGUCUACAGCCAUGGUCGC-3′ | miRNA-132 antisense strand | SEQ ID NO: 8 |
| 5′-UAACAGUCUACAGCCAUGGUCG-3′ | miRNA-132 antisense strand | SEQ ID NO: 9 |
| 5′-UAACAGUCUACAGCCAUGGU-3′ | miRNA-132 antisense strand | SEQ ID NO: 10 |
| 5′-UAACAGUCUACAGCCAUGG-3′ | miRNA-132 antisense strand | SEQ ID NO: 11 |
| 5′-UAACAGUCUACAGCCAUG-3′ | miRNA-132 antisense strand | SEQ ID NO: 12 |
| 5′-UAACAGUCUACAGCCA-3′ | miRNA-132 antisense strand | SEQ ID NO: 13 |
| 5′-UAACAGUCUACAGCCAUGGUCG-3′ | miRNA-132 antisense strand | SEQ ID NO: 14 |
| 5′-ACAGUCUACAGCCAUGGUCG-3′ | miRNA-132 antisense strand | SEQ ID NO: 15 |
| 5′-UAACGGUCUACAGCCAUGGUCG-3′ | miRNA-132 antisense strand | SEQ ID NO: 16 |
| 5′-UAACAGUCUACAGCCAUGGUC-3′ | miRNA-132 antisense strand | SEQ ID NO: 17 |
| 5′-UAACAGUCUACAGCCAUGGUCG-3′ | miRNA-132 antisense strand | SEQ ID NO: 18 |
| 5′-UAACAGUCUACAGCCGUGGUCG-3′ | miRNA-132 antisense strand | SEQ ID NO: 19 |
| 5′-UAACAGUCUACAGCCAUGGUC-3′ | miRNA-132 antisense strand | SEQ ID NO: 20 |
| 5′-AACAGUCUACAGCCAUGGUCG-3′ | miRNA-132 antisense strand | SEQ ID NO: 21 |
| 5′-UAACAGUCUACAGCCAUGGCCG-3′ | miRNA-132 antisense strand | SEQ ID NO: 22 |
| 5′-AACAGUCUACAGCCAUGGUCGCC-3′ | miRNA-132 antisense strand | SEQ ID NO: 23 |
| 5′-UAACAGUCUACAGCCAUGGUCG-3′ | miRNA-132 antisense strand | SEQ ID NO: 24 |
| 5′-UAACAGUCUACAGCCAUGGUCGU-3′ | miRNA-132 antisense strand | SEQ ID NO: 25 |
| 5′-GUCUACAGCCAUGGUCG-3′ | miRNA-132 antisense strand | SEQ ID NO: 26 |
| 5′-UAACAGUCUACAGNCAUGGUCG-3′ | miRNA-132 antisense strand | SEQ ID NO: 27 |
| 5′-UAACAGUCUACAGCCAUGGUCG-3′ | miRNA-132 antisense strand | SEQ ID NO: 28 |
| 5′-UAACAGUCUACAGCCAUGGUC-3′ | miRNA-132 antisense strand | SEQ ID NO: 29 |
| 5′-ACAGUCUACAGCCAUGGUCGC-3′ | miRNA-132 antisense strand | SEQ ID NO: 30 |
| 5′-AACAGUCUCCAGCCAUGGUCGC-3′ | miRNA-132 antisense strand | SEQ ID NO: 31 |
| 5′-UAACAGUCUACAGCCAUGGUCG-3′ | miRNA-132 antisense strand | SEQ ID NO: 32 |
| 5′-CAACAGUCUACAGCCAUGGUCG-3′ | miRNA-132 antisense strand | SEQ ID NO: 33 |
| 5′-ACAGGCUACAGCCAUGGUCGC-3′ | miRNA-132 antisense strand | SEQ ID NO: 34 |
| 5′-UAACAGUCUACAGCCAUGGUCG-3′ | miRNA-132 antisense strand | SEQ ID NO: 35 |
| 5′-NAACAGUCUACAGCCAUGGUCG-3′ | miRNA-132 antisense strand | SEQ ID NO: 36 |
| 5′-GGGCGACCAUGGCUGUAGACUGUUA-3′ | miRNA-132 sense strand | SEQ ID NO: 37 |
| 5′-ACCGUGGCUUUCGAUUGUUACU-3′ | miRNA-132 sense strand | SEQ ID NO: 38 |
| 5′-ACCGUGGCUUUCGAUUGUUAC-3′ | miRNA-132 sense strand | SEQ ID NO: 39 |
| 5′-ACCGUGGCUUUCGAUUGUUA-3′ | miRNA-132 sense strand | SEQ ID NO: 40 |
| 5′-AACCGUGGCUUUCGAUUGUUA-3′ | miRNA-132 sense strand | SEQ ID NO: 41 |
| 5′-ACCGUGGCUUUCGAUUGUU-3′ | miRNA-132 sense strand | SEQ ID NO: 42 |
| 5′-ACCGUGGCUUUCGAUUGU-3′ | miRNA-132 sense strand | SEQ ID NO: 43 |
| 5′-AACCGUGGCUUUCGAUUGU-3′ | miRNA-132 sense strand | SEQ ID NO: 44 |
| 5′-AACCGUGGCUUUCGAUUGUUAC-3′ | miRNA-132 sense strand | SEQ ID NO: 45 |
| 5′-AACCGUGGCUUUCGAUUGUU-3′ | miRNA-132 sense strand | SEQ ID NO: 46 |
| 5′-CCCGUGGCUUUCGAUUGUUAC-3′ | miRNA-132 sense strand | SEQ ID NO: 47 |
| 5′-ACCGUGGCUUUCGAUUG-3′ | miRNA-132 sense strand | SEQ ID NO: 48 |
| 5′-AACCGUGGCUUUCGAUUGUUACU-3′ | miRNA-132 sense strand | SEQ ID NO: 49 |
| 5′-ACCGUGGCUUUCGAUUGUUACC-3′ | miRNA-132 sense strand | SEQ ID NO: 50 |
| 5′-ACCGUGGCUUUCGAUUGUUAC-3′ | miRNA-132 sense strand | SEQ ID NO: 51 |
| 5′-ACCGUGGCUUUCGAUUGUUACU-3′ | miRNA-132 sense strand | SEQ ID NO: 52 |
| 5′-ACCGUGACUUUCGAUUGUUAC-3′ | miRNA-132 sense strand | SEQ ID NO: 53 |
| 5′-AACCGUGGCUUUCGAUUG-3′ | miRNA-132 sense strand | SEQ ID NO: 54 |
| 5′-ACCGUGGCUUUCGAUUG-3′ | miRNA-132 sense strand | SEQ ID NO: 55 |
| 5′-AACCGUGGCUUUCGAUUGU-3′ | miRNA-132 sense strand | SEQ ID NO: 56 |
| 5′-ACCGUGGCUUUCGAUUGUUA-3′ | miRNA-132 sense strand | SEQ ID NO: 57 |
| 5′-ACCGCGGCUUUCGAUUGUUAC-3′ | miRNA-132 sense strand | SEQ ID NO: 58 |
| 5′-NCCGUGGCUUUCGAUUGUUACU-3′ | miRNA-132 sense strand | SEQ ID NO: 59 |
| 5′-CACCGUGGCUUUCGAUUGUU-3′ | miRNA-132 sense strand | SEQ ID NO: 60 |
| 5′-ACCUUGGCUCUAGACUGCUUACU-3′ | miRNA-212 sense strand | SEQ ID NO: 61 |
| 5′-ACCUUGGCUCUAGACUGCUUAC-3′ | miRNA-212 sense strand | SEQ ID NO: 62 |
| 5′-ACCUUGGCUCUAGACUGCUUACUG-3′ | miRNA-212 sense strand | SEQ ID NO: 63 |
| 5′-CCUUGGCUCUAGACUGCUUACUG-3′ | miRNA-212 sense strand | SEQ ID NO: 64 |
| 5′-CCCUUGGCUCUAGACUGCUUACU-3′ | miRNA-212 sense strand | SEQ ID NO: 65 |
| 5′-CCUUGGCUCUAGACUGCUUAC-3′ | miRNA-212 sense strand | SEQ ID NO: 66 |
| 5′-ACCUUGGCUCUAGACUGCUUA-3′ | miRNA-212 sense strand | SEQ ID NO: 67 |
| 5′-CACCCCGCCCGGACACGGAC-3′ | miRNA-212 sense strand | SEQ ID NO: 68 |
| 5′-UAACAGUCUCCAGUCACGGCC-3′ | miRNA-212 antisense strand | SEQ ID NO: 2 |
| 5′-UAACAGUCUCCAGUCACGGCC-3′ | miRNA-212 antisense strand | SEQ ID NO: 69 |
| 5′-UAACAGUCUCCAGUCACGGCCA-3′ | miRNA-212 antisense strand | SEQ ID NO: 70 |
| 5′-UAACAGUCUCCAGUCACGGCCAC-3′ | miRNA-212 antisense strand | SEQ ID NO: 71 |
| 5′-GUAACAGUCUCCAGUCACGGCC-3′ | miRNA-212 antisense strand | SEQ ID NO: 72 |
| 5′-UAACAGUCUCCAGUCACGGCC-3′ | miRNA-212 antisense strand | SEQ ID NO: 73 |
| 5′-UAACAGUCUCCAGUCACGGCCA-3′ | miRNA-212 antisense strand | SEQ ID NO: 74 |
| 5′-UAACAGUCUCCAGUCACGGCC-3′ | miRNA-212 antisense strand | SEQ ID NO: 75 |
| 5′-UAACAGUCUCCAGUCACGGC-3′ | miRNA-212 antisense strand | SEQ ID NO: 76 |
| 5′-UAACAGUCUCCAGUCACGGC-3′ | miRNA-212 antisense strand | SEQ ID NO: 77 |
| 5′-GUAACAGUCUCCAGUCACGGC-3′ | miRNA-212 antisense strand | SEQ ID NO: 78 |
| 5′-UAACAGUCUCCAGUCACGGCCA-3′ | miRNA-212 antisense strand | SEQ ID NO: 79 |
| 5′-UAACAGUCUCCAGUCACGGCCAGA-3′ | miRNA-212 antisense strand | SEQ ID NO: 80 |
| 5′-UAACAGUCUCCAGUCACGUCC-3′ | miRNA-212 antisense strand | SEQ ID NO: 81 |

Preferably, the mimic miRNA is a doubled stranded ribonucleic acid molecule comprising one first strand consisting of SEQ ID NO: 3 and a second strand consisting of SEQ ID NO: 37.

In a preferred embodiment, mimic miRNA encompassed herein comprises a spacer sequence which comprises a sequence consisting of the mature miRNA-132 or -212 sequences 3′ of their respective seed sequence in the native sequence. The mimic miRNA molecule encompassed herein comprises miRNA-132 or miRNA-212 sequence, a precursor or an analog thereof.

In a further embodiment, the mimic miRNA encompassed herein is a doubled stranded RNA molecule, which may comprise at least one modified building block or suitable modified deoxyribonucleotide moieties as known in the art. Modified nucleotide building blocks may be selected from nucleobase-, sugar- and backbone-modified building blocks and combinations thereof, i.e. building blocks having several modifications, e.g. a sugar and a backbone modification.

Nucleobase-modified building blocks encompassed herein comprise a non-standard nucleobase instead of a standard nucleobase (e.g. adenine, guanine, cytosine, thymine or uracil) such as a uracil or cytosines modified at the 5-position, e.g., 5-methylcytosine, 5-(2-amino)propyluracil, 5-bromouracil, adenines or guanines modified at the 8-position, e.g. 8-bromoguanine, deazapurine nucleobases, e.g. 7-deaza-adenine and O- or N-alkylated nucleobases, e.g. N6 alkyl-adenine.

Sugar-modified building blocks, particularly sugar-modified ribonucleotide building blocks encompassed herein, comprises wherein 2′OH group is replaced by a group selected from H, OR, R, halo, SH, SR, NH, NHR, NR₂ or CN, wherein R is C,-C₆ alkyl, C₂-C₆ alkenyl or C₂-0₆ alkynyl and halo is F, Cl, Br or I. Further sugar-modified nucleotides are selected from LNA or morpholino nucleotides.

Encompassed backbone-modified building blocks are phosphodiester group connecting to adjacent building blocks replaced by a modified group, e.g. by replacing one or more O atoms of the phosphodiester group by S, Se, NR or CR₂, wherein R is as defined above. Also encompassed are phosphorothioate deoxyribose groups as the backbone unit; an N′3-N′5 phosphoroamidate deoxyribonucleotide moiety, which comprises an N′3-N′5 phosphoroamidate deoxyribose group as the backbone unit; and/or a ribonucleotide moiety that is substituted at the 2′ position. A substituent at the 2′ position of a modified ribonucleotide moiety can be for example a C1 to C4 alkoxy-C1 to C4 alkyl group. The C1 to C4 alkoxy (alkyloxy) and C1 to C4 alkyl group may comprise any of the alkyl groups described above, such as for example a C1 to C4 alkoxy-C1 to C4 alkyl group consisting of methoxyethyl. Also encompassed is the presence of a methylene bridge between the 2′-oxygen atom and the 4′-carbon atom, a substitution at the 2′ position with fluoro group (such as 2′-fluororibonucleotide moieties known in the art).

Other chemical modifications may include cell-penetrating compounds such as cholesterol or peptides. Examples include penetratin, transportan, and TAT peptides.

Other carriers include, but are not limited to, saline, glucose, and lipid-based solutions (e.g., LipofectAMINE and in vivo-jetPEI).

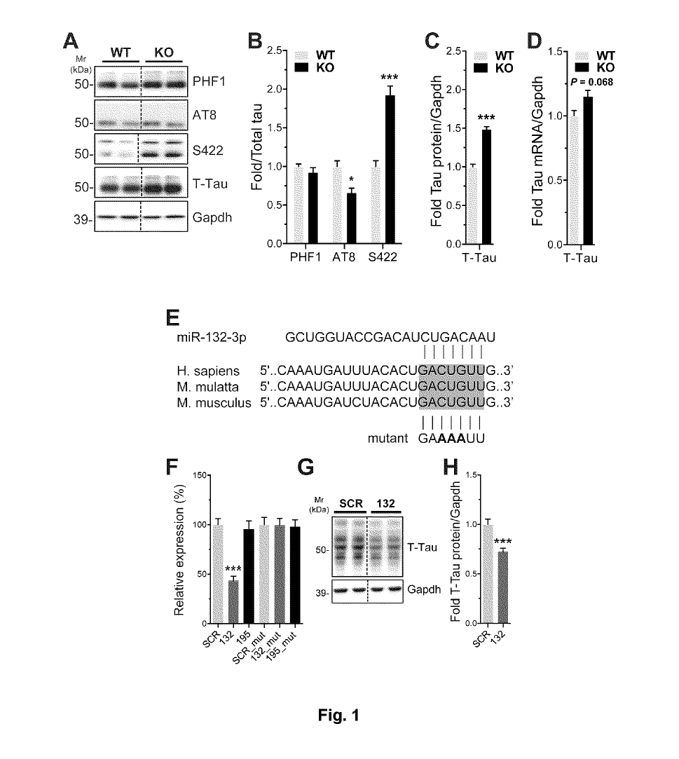

As described herein, increase of specific Tau phosphorylation was observed with the use of anti-phospho S422 in miRNA-132/212 knockout mouse cortex (see FIG. 1b). Although decrease of Tau AT8 phosphorylation signal was detected in miRNA-132/212 KO mouse, total Tau signal was also increased and then, phosphorylation/expression protein ratio was still higher compared to control mice (FIG. 1b). miRNA-132 mimics directly target the 3′ untranslated region of Tau and affects it expression in a luciferase reporter assay and in neuronal cells (see FIG. 1f). This effect is blocked when mutating the miRNA-132 binding site in Tau mRNA.

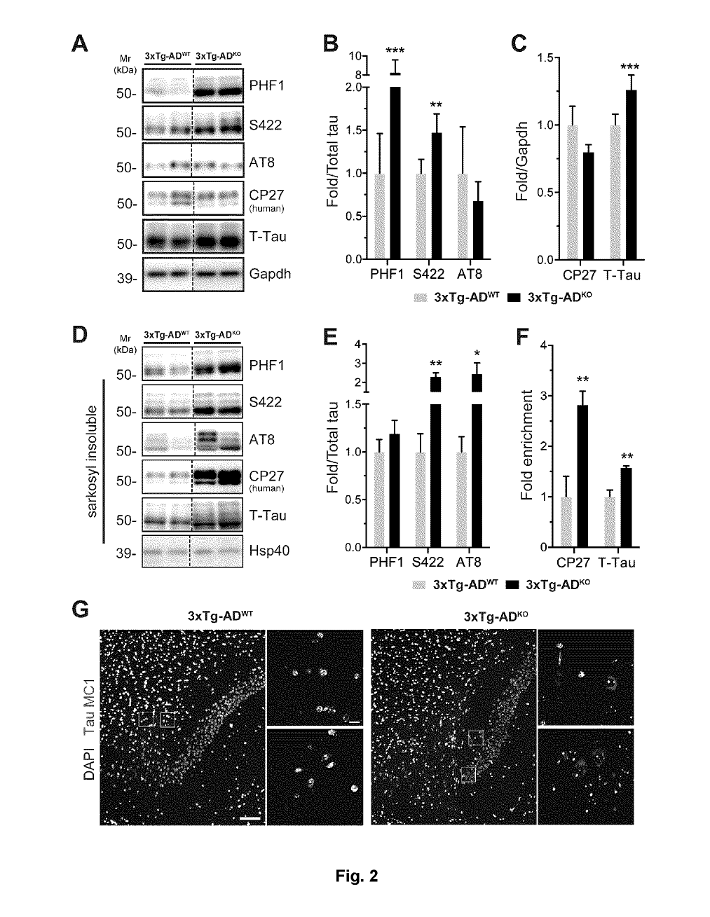

Furthermore, genetic deletion of miRNA-132 and miRNA-212 in the 3×Tg-AD Alzheimer mouse model triggers Tau hyperphosphorylation, expression and aggregation, confirming miRNA-132's role in repressing Tau expression and metabolism. In the soluble fraction, S422, PHF-1 and total Tau signals were significantly increased compared to control 3×Tg-AD mice at 6 months (see FIGS. 2a, b, c), whereas S422, total Tau and CP27 (specific for human total Tau) were increased in the sarkosyl insoluble fraction of 6 month-old mouse brain (FIGS. 2d, e, f) compared to controls. At 18 months, MC1 signal (specific for Tau neurofibrillary tangles) was increased in 3×Tg-AD/miRNA-132/212 KO mice when compared to 3×Tg-AD control mice. Thus, miRNA-132/212 deficiency accelerates and/or exacerbates Alzheimer's disease and Tauopathy pathologies in mammals.

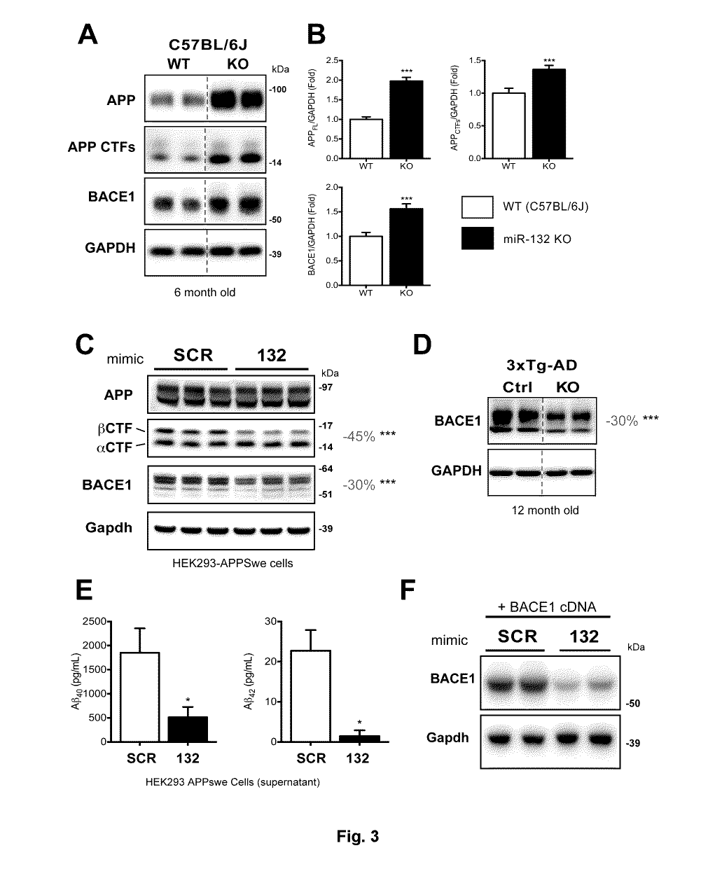

Also evidenced herein is the APP amyloidogenic pathway regulation by miRNA-132/212 (FIG. 3). More particularly, it is confirmed the repressive role of miRNA-132 on BACE1 expression (the rate-limiting enzyme in Aβ production) and Aβ levels. An increase of full length, C-terminal fragments of Amyloid Precursor Protein (APP-FL, APP-CTFs) and BACE1 were detected in miRNA-132/212 knockout mice (see FIGS. 3a and b), as confirmed in an ex vivo cell model (HEK293-APPSwe cells) (FIGS. 3c). miRNA-132 mimic expression can lower Aβ40 and Aβ42 peptides when compared to a scrambled control (SCR) (FIG. 3e, f). Introduction of miRNA-132 mimics reduced cortical BACE1 levels in 12 month-old 3×Tg-AD control mice (FIG. 3d).

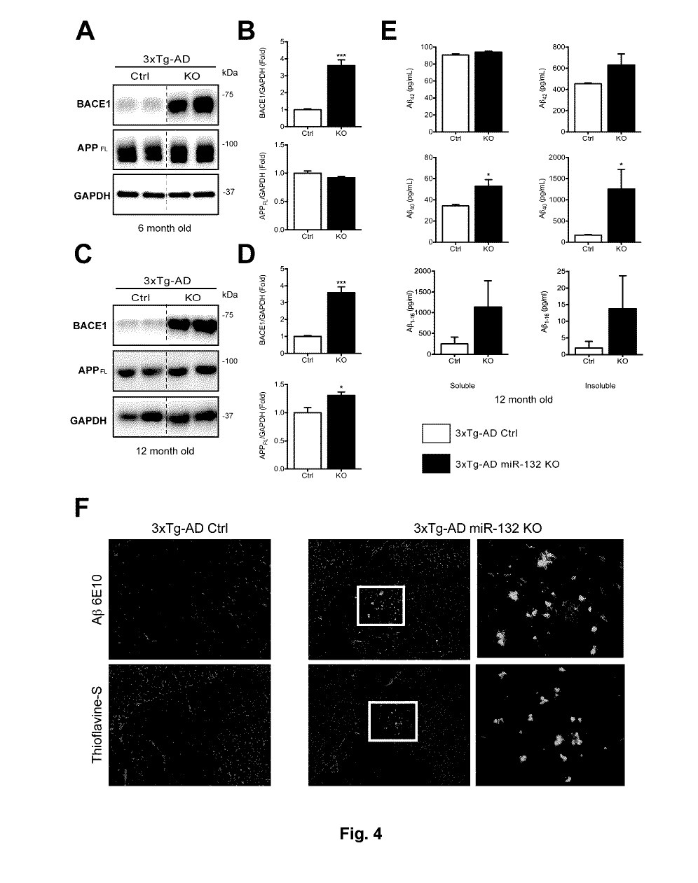

In 3×Tg-AD/miRNA-132/212 KO mice, the APP amyloidogenic pathway was enhanced as evidenced for example by the increase of the β-site APP cleaving enzyme 1 (BACE1) in 6 month-old samples and in 12 month-old mouse cortical homogenates compared to controls (FIGS. 4a-d). APP full length (APP-FL) was changed in 12 month-old 3×Tg-AD mouse samples, and Aβ₄₀ and Aβ₄₂ peptides were increased in both soluble and insoluble fractions from cortex homogenates of 12 month-old animals compared to controls (FIG. 4e). Aβ plaques were increased in 18 month-old 3×Tg-AD/miRNA-132/212 KO mice compared to 3×Tg-AD control mice (FIG. 4f).

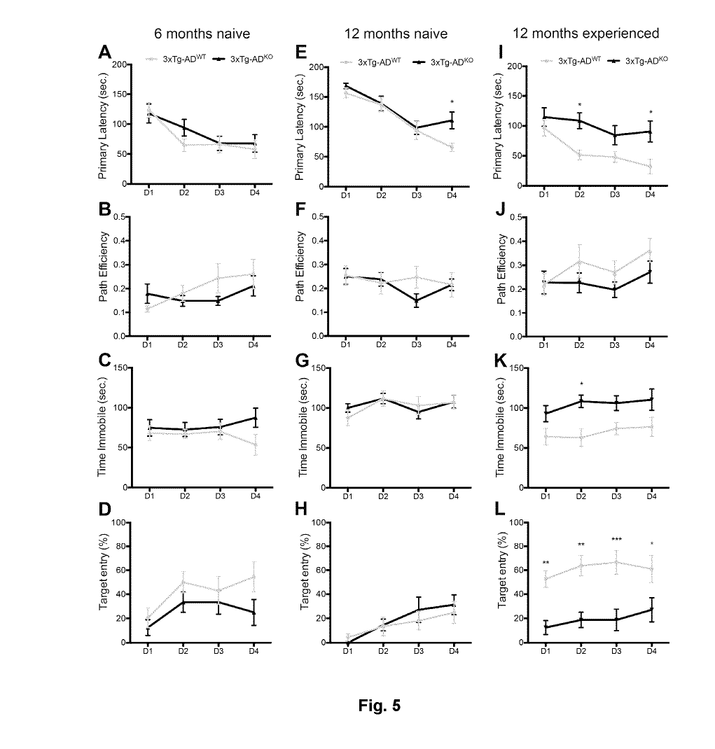

The absence of expression of miRNA-132/212 translated in more cognitive impairment in 3×Tg-AD/miRNA-132/212 KO compared to control 3×Tg-AD control mice at 12 months (FIG. 5).

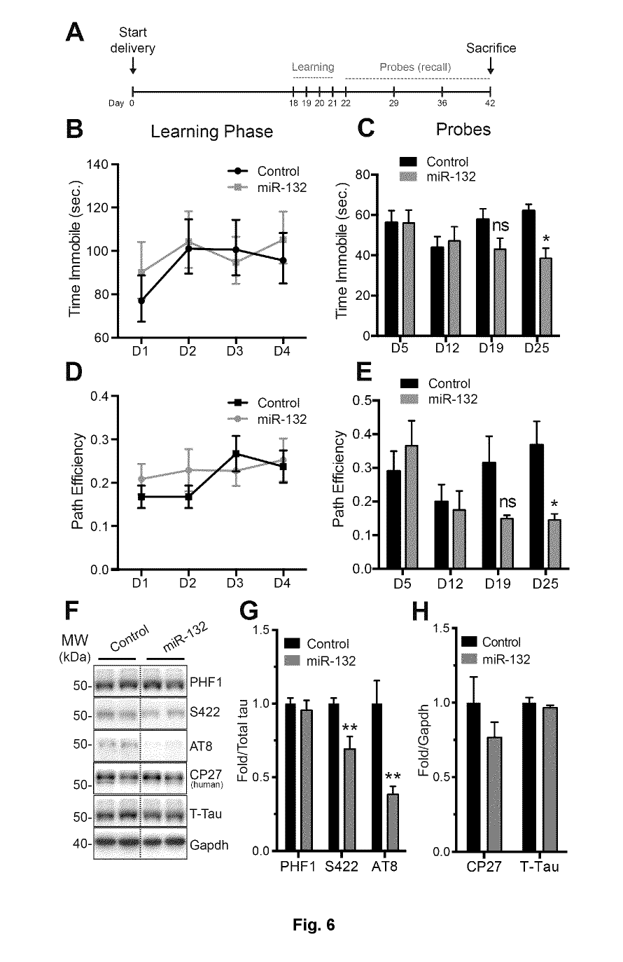

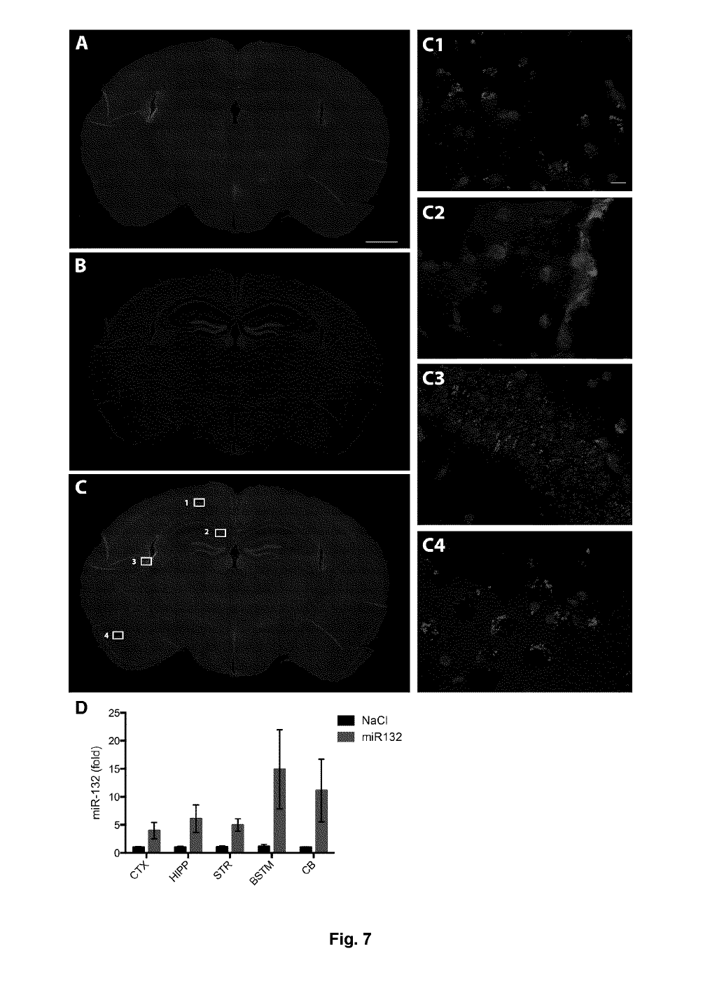

In consequence, in the perspective of reversing the phenotype seen in the absence of expression of miRNA-132/212, 3×Tg-AD mice were injected miRNA-132 mimics as described herein (FIG. 6). A widespread distribution of ectopic miRNA-132 mimics, ranging from 4 to 15 fold increase compared to basal (endogenous) levels was measured (FIG. 7), confirming that the miRNA-132 mimics as described herein was found expressed were it should be, reflecting the stability and specificity of distribution of the mimics described herein.

Further validation of miRNA-132 down regulation in Alzheimer's disease patients is presented (FIG. 8), including in mild cognitive impairment, a prodromal stage of Alzheimer's disease. Such a decrease of miRNA-132 correlated with memory impairments in humans.

The terms ‘pharmaceutical composition’ or ‘medicament’ as used herein relates to a composition comprising a mimic miRNA as described herein to treat neurodegenerative disease. Neurodegenerative diseases or disorder encompassed herein include Alzheimer's disease, tauopathies, Amyotrophic lateral sclerosis, Parkinson's disease, frontotemporal dementia, prion's disease, and Huntington's disease. The composition described herein comprises a suitable carrier or excipient known to the skilled person such as a saline solution, a Ringer's solution, a dextrose solution, a Hank's solution, a fixed oils, an ethyl oleate, a 5% glucose in saline, substances that enhance isotonicity and chemical stability, buffers and preservatives for example.

The composition or medicament described herein can be administered to a mammal by any method known to those skilled in the art. Some examples of suitable modes of administration include oral and systemic administration. Systemic administration can be enteral or parenteral.

Parenteral administration of the medicament includes, for example intravenous, intramuscular, and subcutaneous injections. For instance, a molecule may be administered to a mammal by sustained release, as is known in the art. Other routes of administration include oral, topical, intrabronchial, or intranasal administration.

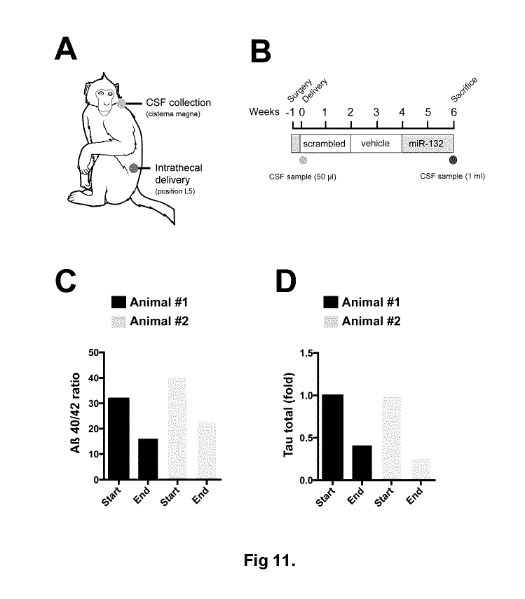

In an embodiment, the composition or medicament is administered intrathecally, by surgically implanting a pump and running a catheter to the spine for example. Intrathecal delivery of miRNA-132 mimics modified Aβ40, Aβ42, and total Tau CSF profiles in non-human primates (FIG. 11).

In another embodiment, the composition or medicament is delivered intracerebroventricularly (ICV), following a surgical intervention to position the pump.

In an embodiment, the composition or medicament encompassed herein further comprises other natural or synthetic compounds that can be used to increase endogenous miRNA-132 or miRNA-212 expression levels in the central nervous system. Examples include Leptin or Luteolin.

In an embodiment, miRNA-132 or miRNA-212 mimics can be used in a combination therapy with current or future medications, including, but not limited to, cholinesterase inhibitors, memantine, vitamin E, anti-Aβ antibodies, omega-3 fatty-acids, or stem cells.

In an embodiment, the effects of miRNA-132 mimics on BACE1 and Tau in vitro and in vivo were specific, and not observed using other miRNANA mimics (e.g., scrambled, miRNA-195, or miRNA-16-modified) (FIGS. 1, 3, 9).

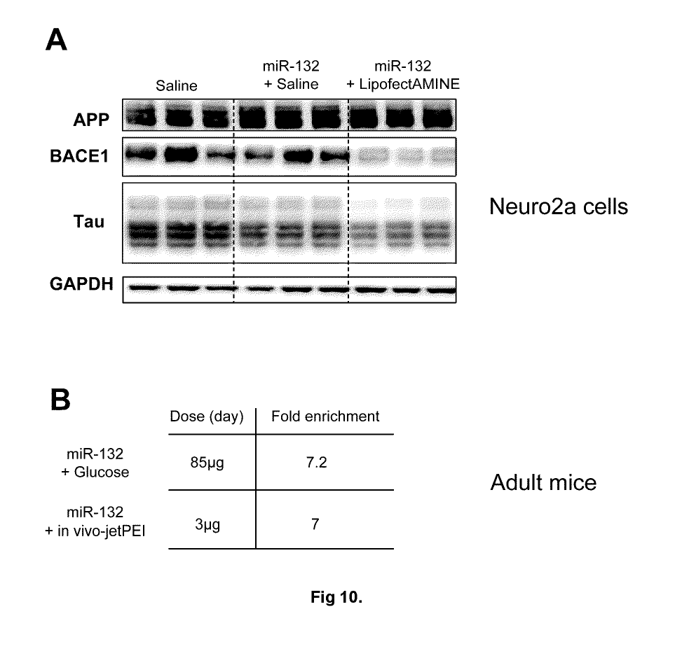

In an embodiment, miRNA-132 mimics display different efficiencies when diluted in carriers (FIG. 10). The use of a carrier, such as for example and not limited to, saline, LipofectAMINE®, glucose, or in vivo-jetPEI® provided a mean to introduce mimics into neuronal cells in vitro or in vivo (see FIG. 10b).

In an embodiment, the stabilizing RNAi-cap™ sequence protects double-stranded oligonucleotides against degradation, including in, but limited to, humans and monkey CSF.

The present disclosure will be more readily understood by referring to the following examples which are given to illustrate embodiments rather than to limit its scope.

### EXAMPLE I

### miRNA-132/212 Knockout Mice and Testing

miRNA-132/212 knockout mice were generated as previously described (Magill et al., 2010, Proc Natl Aca Sci, 107: 20382-20387) and were kindly provided by Dr. Richard H. Goodman (Vollum institute, Portland, Oreg.). miRNA-132/212 knockout(−/−) mice were backcrossed with C57BL/6 for 8 generations. In all studies, littermate controls were used (N=8-12/genotype). The 3×Tg-AD/miRNA-132/212 knockout mice were generated by crossing homozygous miRNA-132 KO mice with 3×Tg-AD homozygous mice to produce quadruple heterozygous Tg litters. Subsequently, heterozygous mice were bred to obtain littermates homozygous for the four transgenes. All animal procedures were conducted according to the Canadian Council on Animal Care guidelines, as administered by the Laval University Animal Welfare Committee.

The Barnes test was used to measure spatial memory of 3×Tg-AD control and 3×Tg-AD/miRNA-132/212 knockout mice. The maze consisted of a metallic and circular disk with 20 holes with equal distance from each other at the edge of the maze. One hole is used to be the escape where the mice can hide in a metallic box beneath the maze. For aversive stimulations, mice were subjected to light (800 lux) and noise (74 dB). A tracking camera device connected to a computer was positioned over the maze to capture behavioral experiments. ANY-maze software (Stoelting Co., Wood Dale, Ill. USA) was utilized as video tracking system. The protocol is designed over 5 days; 4 days with escape box (learning phase) and 1 day without (probe). Each trial last no more than 180 seconds except for the probe (90 sec.). Four trials/day/mouse were done. 24 hours before Day 1, the mice were housed individually and bring to behavioral test room. The next morning, before the first trial, an acclimatization/exploration of 90 seconds (without recording) was done for each mouse. Observed parameters were Primary Latency (time in seconds between the test start and the escape hole), escape probability (escape entry probability in percentage), Immobility while testing in seconds.

### EXAMPLE II

### Ex Vivolin Vitro Testing

Human HEK293-APPswe (constitutively overexpressing the Amyloid precursor protein KM670/71NL mutation) and mouse N2A cells were cultured using Dulbecco's Modified Eagle's Medium (DMEM) supplemented with 10% Foetal Bovine Serum and were maintained in a 5% humidified incubator at 37° C. 75,000 of HEK293 or N2A cells were plated in 24-well plates 24 hours before transfection. 50 nM of miRNA-132 mimics were co-transfected with the full length Mapt mouse 3′UTR luciferase vector (GeneCopoeia, Rockville, Md., USA). Scrambled miRNA mimic or miRNA-195 mimic (Ambion, Life Technologies) were used as negative controls. Mutagenesis was performed by TOP Gene Technologies (Montreal, Quebec, Canada) and confirmed by sequencing. miRNA-132 seed binding region was mutated from GACTGTT to GAAAATT within mouse Mapt 3′UTR plasmid. Twenty-four hours after transfection, measurements were done with the Dual-luciferase® reporter assay kit (Promega, Madison, Wis., USA).

250,000 HEK293 cells were plated in 6 well plates 24 hours before transfection. 50 nM of miRNA-132 mimics and scrambled mimic were co-transfected with a pCDNA3 vector expressing the coding sequence region of human BACE1. Forty-eight hours post-transfection, cells were harvested to obtain protein lysates for immunoblot analysis.

250,000 N2A cells were plated in 6 well plates the day before transfection. 50 nM of miRNA-132 mimic (Riboxx Life Sciences, Germany) or 50 nM of scrambled mimic (control) (Ambion) were transfected for 48 hours.

For mouse brain tissues, mice were killed by decapitation without anesthesia and perfusion. Brains were immediately removed, dissected and frozen on dry ice and stored at −80° C. until use. Protein extracts were done by mechanically homogenizing the brain tissues in approximately 5× volume/weight RIPA buffer containing 50 mM Tris-HCl pH 7.4, 1 mM ethylenediaminetetracetic acid (EDTA), 150 mM NaCl, 0.5% sodium deoxycholate, 1% Octyl-Phenyloxypolyethoxyethanol (IGEPAL CA630, substitute of NP-40), 1 mM phenylmethylsulfonyl fluoride (PMSF), 1 tablet (per 50 ml RIPA) of Complete ETDA-free Protease Inhibitor Cocktail (Roche life Science), 1 mM activated sodium Orthovanadate, 2 mM sodium Fluoride (NaF), 15 nM okadaic acid (OA, Sigma-Aldrich). Then, samples were sonicated for 10-15 s in pulse mode and incubated for 20-30 min. on ice and centrifuged at 20,000 g for 20 min. at 4° C. Protein concentration was determined in the sample supernatant using Bradford method (Bio-Rad Protein Assay).

Soluble RIPA homogenates were treated with 1% N-Lauroylsarcosine sodium salt (Sarkosyl, Sigma-Aldrich). Samples were incubated with rotation for 1 hour at 37° C. and centrifuged at 100,000 g for 1 hour at 20° C. in a Sorvall™ MTX 150 Micro-Ultracentrifuge (Thermo Scientific). Supernatants were discarded and the remaining pellets were resuspended in 1× LDS Sample Buffer (Life Technologies) for further Western immunoblot analysis.

Protein lysates were extracted from cell cultures using the RIPA buffer described above. Cells were rinsed with PBS and harvested in 100 μl of RIPA with a sterile cell scraper. Then, soluble lysates were processed as above.

Total RNA was extracted from brain tissues using TRIzol® Reagent (Life Technologies) according to the manufacturer's instructions. RNA concentrations were determined using NanoQuant plate™ with the Infinite F200 microplate reader (Tecan Group Ltd., Switzerland). Mature microRNAs were reverse transcribed with the TaqMan® MicroRNA Reverse Transcription Kit (Life Technologies) and amplified with probe-specific TaqMan® miRNA assays (Life Technologies) using LightCycler® 480 (Roche life Science) according to the manufacturer's instructions. Relative expression was calculated by using the comparative delta CT method (2-ddCT). RNU19 probe was used as normalization control.

Protein expressions included in this study were determined using SDS-polyacrylamide gel electrophoresis (PAGE) followed by Western immunoblotting analysis. Protein lysates from mice tissues (10 μg) or cell cultures (30 μg) were loaded and separated on a 10% Tris-Glycine SDS-PAGE and transferred onto lmmobilon-P Polyvinylidene fluoride (PVDF) transfer membranes (EMD Millipore). Tris-buffered saline containing 0.1% Tween 20 was used to block non-specific binding sites for 1 hour at room temperature. Antibodies were incubated overnight at 4° C. Antibodies used were: Tau phosphoSerine 422 (S422, EMD Millipore); PHF1 (Peter Davies); Total Tau (Dako); GAPDH (EMD Millipore); CP27 (Peter Davies); APP C-ter (Sigma-Aldrich); BACE1 (Cell Signaling); and were diluted as recommended. Membranes were washed 3 times and then incubated for 1 hour at room temperature with a horseradish peroxidase-linked secondary antimouse or rabbit antibody (Jackson ImmunoResarch laboratories). Immunoreactivity was detected using Immobilon™ Western ECL (EMD Millipore) and were visualized with the Fusion FX5 Imaging system (Vilber Lourmat). Image J software was used to analysis densitometric band intensities.

Aβ peptides from the mouse cortex were homogenized to obtain the soluble and the insoluble Aβ fractions. Then, the pellet (insoluble Aβ) was resuspended with RIPA buffer and transfer into ultracentrifugation tube and centrifuged at 100,000 g for 20 minutes at 4° C. in a Sorvall™ MTX 150 Micro-Ultracentrifuge (Thermo Scientific). The further pellet was saved, dissolved in 99% formic acid (Sigma-Aldrich) and was gently homogenized or sonicated. The resulting homogenates were centrifuged at 10,000 g for 20 minutes at 4° C. The supernatant were gathered and placed under chemical hood until the formic acid was completely evaporated. After, dried supernatants were resuspended in 5M-guanidine buffer and stored at 4° C. before ELISA.

After 48 post-transfection, 1 mL of DMEM was removed per well from transfected HEK293-APPswe cells (miRNA-132 mimic; SEQ ID NO: 1; or scrambled control) and centrifuged 5-10 min. at 10,000 g at 4° C. Supernatants were kept to performed Aβ quantifications. To measure soluble and insoluble Aβ40, Aβ42, Aβ oligomers peptides, Human amyloid-β (Life Technologies, USA), human amyloid-β 42 (Life technologies, USA) and Human amyloid-β oligomers (82E1-specific) (IBL International, Japan) assay kits were used respectively following manufacturer's protocols. ELISA plates were read at 450 nm with the Infinite F200 microplate reader (Tecan Group Ltd., Switzerland).

Brains were fixed with 4% paraformaldehyde for 72 h and embedded in paraffin. Five-micrometer serial sagittal sections were collected with a microtome. Slices were rehydrated, incubated in blocking solution (7.5% NGS; 0.4% Triton; 1% BSA; PBS) for 2 h and in MC1 antibody solution (5% NGS; 0.4% Triton; PBS) overnight. Slices were incubated 2h 30 in the secondary antibody solution (AlexaFluor 568, #A11031; Invitrogen) and 5 min in 30 nM DAPI. Slices were observed using a Zeiss Axiolmager M2 microscope under 20× and 63× magnification, and images were processed with a computerized image analysis system (ZEN 2012 SP2 Software, Zeiss).

### EXAMPLE III

### Mimics of miRNA-132/212 Administered to Mice

miRNA-132 mimic delivery to the mouse brain was achieved by stereotactic injection and a mini-pump system. Twelve-month-old wildtype 057/BI6 or 3×Tg-AD control mice were implanted with mini-pumps (ALZET® model 2006, USA) according to the manufacturer's instructions. Brain infusion kits were purchased from Durect (Denmark). Pre-operative procedure included 30 μl of Anafen (1 mg/ml), 100 μl Marcaine (5.0 mg/ml), and 500 μl saline (0.9%). miRNA-132 mimics or miRNA-16-modified mimics were administrated into the brain (coordinates: ventricle A/P=−0.22 M/L=0.0 D/v=−3.5) for 7 or 42 days at a rate of 1.8-3 μg/day (N=5-10). Five percent (5%) final volume of in vivo-jetPEI® (Polyplus®, France) was added to the mixture before delivery. Mice treated with vehicle alone (glucose+in vivo jetPEI) served as negative controls (N=5-11). During the post-operative procedure, mice were treated with 50 μl Anafen (1 mg/ml) and 500 μl saline (0.9%). Mice were sacrificed without anesthesia 7 or 42 days post-injection. Fluorescent miRNA-132 mimics (with 5′RED555 fluorescent conjugation) were used in a subset of mice.

### EXAMPLE IV

### Mimics of miRNA-132/212 Administered to Non-Human Primates

Cynomolgus monkeys (Macaca fascicularis, 3-4 years old, males) were used. Animals were kept in cages according to the Canadian Council on Animal Care (CCAC) guidelines, and maintained in a high nutritional and stimulating environment (toys, fruit, television, etc.). Monkeys were fed using standard food (Harlan Teklad Diet/primate diet) and receive daily observations.

miRNA-132 mimic delivery into the nonhuman primate brain was achieved using a mini pump system (e.g., model iPRECIO, ALZET Osmotic Pumps, USA). Animals were placed under anesthesia and received a general analgesic. The puncture site (position L4-L6) received an additional local analgesic, and was then aseptised and cut open. A small incision was performed in the dura matter (position L5) to release the cerebrospinal fluid (CSF). A catheter was inserted while taking care not to harm the spinal cord. The catheter was then fixed and attached to a pump (ALZET® model iPRECIO). The pump was finally stored under the skin, and the wound was closed and sewed. It was possible to insert a second catheter into the cisterna magna with a subcutaneous port. This common procedure allowed us to collect CSF in awakened animals. After surgery, the monkeys were awakened following standard post-operation procedures.

The pump was filled with miRNA-132 mimics and a carrier (vivo-jetPEI). Control animals receive either the carrier alone (e.g., 5% glucose) or a control oligonucleotide (e.g., scrambled). Monkeys received a dose of 54 μg mimics per day for 28 days. During this time, blood was collected every 3-4 days in awakened animals. CSF was collected before and after treatment.

CSF Aβ and Tau profiles were measured before and after miRNA-132 mimic delivery. This was done using commercial ELISA kits. Epitopes included Aβ1-40, Aβ1-42, and Tau total.

While the invention has been described in connection with specific embodiments thereof, it will be understood that it is capable of further modifications and this application is intended to cover any variations, uses, or adaptations of the invention, including such departures from the present disclosure as come within known or customary practice within the art to which the invention pertains and as may be applied to the essential features hereinbefore set forth, and as follows in the scope of the appended claims.

1. A pharmaceutical composition comprising:
a doubled stranded ribonucleic acid molecule comprising a seed region sequence of miRNA-132 or miRNA-212, a spacer, and a stabilizing sequence; and
a carrier.

2. The pharmaceutical composition of claim 1, wherein the spacer sequence comprises a sequence 3′ of the miRNA-132 or -212 seed sequence.

3. The pharmaceutical composition of claim 1, wherein the stabilizing sequence consists of the RNAi-cap™ sequence.

4. The pharmaceutical composition of claim 1, wherein said doubled stranded ribonucleic acid molecule comprises one strand consisting of SEQ ID NO: 1 to 81.

5. The pharmaceutical composition of claim 1, wherein said doubled stranded ribonucleic acid molecule comprises one strand consisting of SEQ ID NO: 3 and a second strand consisting of SEQ ID NO: 37.

6. The pharmaceutical composition of claim 1, wherein said doubled stranded ribonucleic acid molecule further comprises uracil.

7. The pharmaceutical composition of claim 1, wherein said doubled stranded ribonucleic acid molecule further comprises at least one cytosine modified at the 5′-position.

8. The pharmaceutical composition of claim 1, wherein said doubled stranded ribonucleic acid molecule further comprises methylcytosine, 5-(2-amino)propyluracil, 5-bromouracil, 8-bromoguanine, 7-deaza-adenine or N6 alkyl-adenine.

9. The pharmaceutical composition of claim 1, wherein said doubled stranded ribonucleic acid molecule further comprises at least one sugar-modified block, at least one sugar-modified ribonucleotide building block, at least one of LNA nucleotide, at least one morpholino nucleotide, at least one phosphodiester group, at least one phosphorothioation modification, at least one phosphoroamidate deoxyribonucleotide moiety, at least one ribonucleotide moiety that is substituted at the 2′ position.

10-15. (canceled)

16. The pharmaceutical composition of claim 1, wherein said doubled stranded ribonucleic acid molecule further comprises at least one methylene bridge or at least one ribonucleotide moiety that is substituted at the 2′ position.

17. (canceled)

18. The pharmaceutical composition of claim 1, further comprising cholesterol, penetratin, transportan, or TAT peptide.

19. The pharmaceutical composition of claim 1, wherein said carrier is a saline solution, glucose, or a lipid-based solution.

20-24 (cancelled)

25. The pharmaceutical composition of claim 1, further comprising a natural or synthetic compound.

26. The pharmaceutical composition of claim 25, wherein the natural or synthetic compound increases endogenous miRNA-132 or miRNA-212 expression levels in the central nervous system.

27. The pharmaceutical composition of claim 25, wherein the natural or synthetic compound is Leptin or Luteolin.

28. The pharmaceutical composition of claim 1, further comprising an anti-Alzheimer's compound.

29. The composition of claim 28, wherein said anti-Alzheimer's compound is Donepezil, rivastigmine, galantamine, a cholinesterase inhibitor, memantine, vitamin E, an anti-Aβ antibody, an omega-3 fatty-acid, or stem cells.

30-34. (canceled)

35. A method for treating a neurodegenerative disorder in a patient comprising administering to said patient the composition of claim 1 to said patient.

36-37. (canceled)

38. The method of claim 35, wherein said neurodegenerative disorder is Alzheimer's disease, tauopathies, Amyotrophic lateral sclerosis, Parkinson's disease, frontotemporal dementia, prion's disease, mild cognitive impairment or Huntington's disease.

39. The method of claim 38, wherein said neurodegenerative disorder is Alzheimer's disease.

40-44. (canceled)
